(19) United States
(12) Patent Application Publication
(10) Pub. No.: US 2024/0368666 A1
(43) Pub. Date: Nov. 7, 2024

(54) A METHOD AND SYSTEM FOR DETERMINING A QUANTITATIVE COMPOSITION RATIO OF A MICROBIAL STRAIN MIXTURE FOR USE IN A FERMENTATION PROCESS

(71) Applicant: DSM IP Assets B.V., HEERLEN (NL)

(72) Inventors: Willi GOTTSTEIN, Echt (NL); Paul KLAASSEN, Echt (NL); Paulus Petrus DE WAAL, Echt (NL)

(21) Appl. No.: 18/291,626

(22) PCT Filed: Jul. 26, 2022
(86) PCT No.: PCT/EP2022/070992

§ 371 Date: Jan. 24, 2024

(30) Foreign Application Priority Data
Jul. 26, 2021 (EP) 21187737.8

(51) Int. Cl. C12Q 1/06; A23C 9/123

(52) U.S. Cl. CPC C12Q 1/06; A23C 9/123

(57) ABSTRACT

A method of determining a quantitative composition ratio of a microbial strain mixture for use in a fermentation process. The method includes repeatedly performing method cycles, each method cycle comprising the steps of: selecting a set of plurality of microbial strain mixtures having different quantitative composition ratios; concurrently performing fermentation processes for each selected microbial strain mixture; determining, for each of the fermentation processes, at least one performance indicator of the microbial strain mixture; and providing the at least one performance indicator for each of the fermentation processes to a statistical model which is configured to optimize a predefined objective function, wherein the statistical model is configured to determine, based on said determined performance indicators, a subsequent set of plurality of microbial strain mixtures with different quantitative composition ratios for selection in a next cycle.

### FIELD

The invention relates to a method and system for determining a quantitative composition ratio of a microbial strain mixture for use in a fermentation process. The invention further relates to a method for producing a fermented food product. Additionally, the invention relates to a computer program product.

### BACKGROUND

Determining novel microbial strain mixtures for use in fermentation processes for the production of fermented food products is known to be a difficult task. In addition, it entails multiple rounds of trial and error. Also for this reason, the process is often slow and expensive. The microbial strain mixtures have a particular composition of individual strains selected from a larger group of strains. The microbial strain mixture can have a specific quantitative composition ratio determined by the relative abundances of the individual strains. Many times a strain mixture necessary for resulting in a fermented food product with desired properties doesn't exist. Other times, a mixture exists but performs sub-optimally concerning the one or more desired properties. In other cases, strain mixtures are desired, consisting of different strains, but divided across multiple blends with the same desired properties relative to each other (so-called phage rotations). In these situations, it is necessary to be able to fine tune the microbial strain mixture composition ratios to change its behavior in the fermentation process such that it can provide the fermented food product with desired properties.

A small group of microbial strains can result in a large number of possible variations. As a result, typically only a limited number of strains, for example smaller than 5, are used for making novel strain mixtures due to the complexity of the design space. Using a larger number of strains may make the design space vast, making it very challenging to exhaustively search with limited time and resources.

Because the number of possible strain combinations is so vast and the knowledge of strain design space so limited, the amount of time it takes to find better strain mixtures can be the limiting factor in achieving reasonable product development periods. There is a strong desire to efficiently discover new and better strain mixtures because enhanced strain mixtures can have a high impact on the properties of a fermented food product and productivity directly, which impacts profitability. Furthermore, often, the current methods for discovering better microbial strain mixtures rely on low-throughput measurement techniques.

### SUMMARY

It is an object of the invention to provide for a method and a system that obviates at least one of the above mentioned drawbacks.

Additionally or alternatively, it is an object of the invention to provide an objective and/or systematic way of designing new strain mixtures for use in the production of fermented food products.

Additionally or alternatively, it is an object of the invention to provide for a more efficient process for determining of a quantitative composition ratio of a microbial strain mixture for use in a fermentation process for producing a fermented food product.

Thereto, the invention provides for a method of determining a quantitative composition ratio of a microbial strain mixture for use in a fermentation process, the microbial strain mixture including at least two microbial strains selected from a group of microbial strains, wherein the method includes repeatedly performing method cycles, each method cycle comprising the steps of: selecting a set of plurality of microbial strain mixtures having different quantitative composition ratios; concurrently performing fermentation processes for each microbial strain mixture of the selected set of plurality of microbial strain mixtures having different quantitative composition ratios, wherein the fermentation processes in each cycle involve culturing a food product with each microbial strain mixture of the selected set of plurality of microbial strain mixtures for obtaining a fermented food product; determining, for each of the fermentation processes, at least one performance indicator of the microbial strain mixture; and providing the at least one performance indicator for each of the fermentation processes to a statistical model which is configured to optimize a predefined objective function, wherein the statistical model is configured to determine, based on said determined performance indicators, a subsequent set of plurality of microbial strain mixtures with different quantitative composition ratios for selection in a next cycle.

The selection of a strain mixture will have significant effect on the properties of the fermented food product. The method and system allow for an efficient tuning of the strain mixture in order to obtain fermented food products with desired properties. The invention provides an efficient way to determine particular strain mixtures to obtain a desired fermented food product, even when a large number of strains can be combined. The method can provide for an efficient automated culture design.

The method may iteratively determine one or more strain mixtures with specific quantitative composition ratios which lead to certain properties in the fermented food product at or close to desired/targeted properties. Advantageously, the properties of the resulting fermented food product can be improved iteratively, converging to the desired/targeted properties of the fermented food product. Hence, the statistical model may iteratively propose or recommend new strain mixtures with particular strain composition ratios which reduce the difference between the actual properties of the fermented food product using said new strain mixtures and the desired/targeted functional properties of the fermented food product.

A score may be calculated based on the performance indicators. An objective function can be defined, which is a function of the preselected performance indicators which are monitored. Hence, a score may be determined for each of the experiments. The statistical model can be used to link the inputs (cf. combination and ratios of strains) to the output (cf. score). The statistical (machine learning) model may propose which new combination and/or ratios of strains are to be used in the next round.

The statistical model may initially explore the parameter space in order to learn. Then, after a number of iterations, knowledge of the parameter space can be exploited in order to determine microbial strain mixtures which result in enhanced properties in the fermented food product. The objective function can define desired ranges of performance indicators. For example, lower limits and upper limits can be defined using the ranges.

Optionally, the group of strains includes n strains, wherein different strains and different amounts are selected every iteration by means of the statistical model based on predefined product properties. Every selected strain mixture may result in different product properties.

Advantageously, the probability of major phage issues, for example in the production of dairy products, can be reduced by avoiding suboptimal choices using the same strain in more than one phage rotation. Instead, the method allows using a group of a large number of strains, and determine new strain mixtures providing the same or similar desired product properties, thereby reducing the risk of major phage issues.

In an example, a group of strains is provided with multiple different strains that can be used to compose microbial strain mixtures/blends. Several microbial strain mixtures are assembled using certain inoculation dosages per strain, the respective media components are added, and the microbial strain mixture is fermented and the resulting performance indicators are measured. The performance indicator of each blend is evaluated using an objective function which is a function of the available performance indicators. A machine learning framework may be used to link the input, i.e. the inoculation dosages and media components, to the output, i.e. the value of the objective function, in order to ultimately predict microbial strain mixtures to be tested in the next round of experiments, i.e. a next method cycle. Multiple method cycles may be successively repeated. For example, this procedure can be repeated until (i) a maximum number of iterations/cycles has been reached, (ii) the objective value reached its desired value or (iii) if no further improvement could be obtained for a specified number of consecutive cycles. A Gaussian process may be used to fit the model, maintaining a measure of uncertainty.

The concurrent fermentation processes performed in each cycle involve culturing a food product with each microbial strain mixture of the selected set of plurality of microbial strain mixtures for obtaining a fermented food product. Each microbial strain mixture as used herein refers to a different microbial mixture selected from the set of plurality of microbial strain mixtures.

Optionally, the method cycles are repeated one or more times until at least one optimized microbial strain mixture is identified which when used in fermenting of a food product results in a fermented food product having at least one desired functional property.

In some embodiments, the method cycles are repeated at least three times, preferably at least five times, or even at least ten times, wherein in each method cycle, a set of at least five, preferably at least ten, microbial strain mixtures having different quantitative composition ratios is selected. Providing a series of cycles, each having with parallel experiments, with compositional optimization between individual cycles enable to probe a large parameter space with a comparatively reduced total experimental effort, e.g. as compared to designs with random compositional variations between cycles or experimental designs aiming to a cover the parameter space with only parallel experiments,

Optionally, the method is used for targeted strain mixture design. In some examples, certain desired properties in an already optimized strain mixture can be added/removed by means of the objective function.

The statistical model can be configured to determining/recommending experiment designs in each successive method cycle. The method cycles may be repeated until a certain number of iterations have been carried out and/or until a certain condition is met, for example when one or more mixtures are usable for obtaining the fermented food product with desired/targeted properties.

In yet other of further embodiments, the statistical model can be configured to determine the subsequent set of plurality of microbial strain mixtures with different quantitative composition ratios for selection in a next cycle, based on comparing said determined performance indicators with a respective reference value or range.

The respective reference value or range can be any one or more of the reference value, ranges or performance indicators as disclosed herein such as one or more of a viscosity value or range, a time to reach (TTR), a pH value or range, and/or a pH value or range during shelf-life (also referred to as post-acidification or PA). A time for evaluating shelf-life may be reduced by evaluating acidity of a sample kept at 20° C. over a pre-defined time period. The PA-value after ten days (at 20° C.) was found to be a useful to compare shelf-life.

In one example the one or more performance indicators may be weighed, e.g. with respect to a deviation from a target value or range. In some examples the weighing may be non-linear, e.g. progressive or exponential, as a measured indicator value increasingly deviates from a target value. As such weighing the deviation may contribute to speeding up a conversion of the selection process towards the desired value.

Optionally, the statistical model is a Bayesian optimization model wherein the objective function to be optimized is approximated by a Gaussian process.

Advantageously, the Bayesian model can work with relatively small sample sizes compared to other methods. In various examples, other machine learning models, such as reinforcement learning, may require extensive resources, as millions of experiments may be required for optimization. In this case, each experiment may require physical actions, for example using a robotic arrangement, for which the Bayesian model may be more suited. Additionally, the Bayesian model can use prior knowledge. Hence, experimental data from other optimization campaigns can be easily used, providing an even more efficient optimization. The Bayesian model can be used to build upon available historical data. Hence, this provides a way to fully leverage the prior knowledge. Additionally, all kind of production data generated in the past can be used, to speed up the process. Genetic algorithms, for instance, typically use the previous generations basically. By taking more data into account, the accuracy and/or efficiency of determining enhanced microbial strain mixtures can be improved.

Working with a relatively small sample sizes is particularly advantageous in optimization of process comprises lengthy and/or costly performance evaluations, such as determination of shelf-life. In some preferred embodiments, one or more reference strains are included in at least some, optionally all, of the subsequent process cycles. Inclusion of one or more reference microbial strain mixture in subsequent process cycles advantageously allows the model to account for noise between process cycles such as variations due to composition and/or quality of substrates. Correction for noise can be of particular relevance for optimization cycles having a process time that is much larger than a typical shelf-life of one or more of the constituents used in the process, such as the substrate for the microbial strains (e.g. varying milk consistency). Using a reference strain/blend with known performance can thus mitigate noise between subsequent process cycles. Accordingly, in some embodiments, in one or more, preferably each method cycle, the selected set of plurality of microbial strain mixtures includes a reference microbial strain mixture having a predetermined composition ratio of microbial strain mixtures; the fermentation process is concurrently performed for each microbial strain mixture of the selected set of plurality of microbial strain mixtures including the reference microbial strain mixture; at least one reference performance indicator of the reference microbial strain mixture is determined; and the at least one reference performance indicator is provided to the statistical model to optimize the predefined objective function, wherein the statistical model is configured to determine the subsequent set of plurality of microbial strain mixtures with different quantitative composition ratios for selection in a next cycle based at least in part on comparing the performance indicators of the different mixtures, respectively, with a value of the at least one reference performance indicator of the reference microbial strain mixture as determined in the same method cycle.

In other or further embodiments, for each method cycle, at least some, preferably most, or even all, of the microbial strain mixtures are determined with a quantitative composition ratio that is different from the composition ratio of any microbial strain mixtures determined in any preceding method cycle.

A fermented food product such as yogurt can be produced by using a mixture/blend of strains selected by performing multiple method cycles according to the method. In the selected mixture of microbial strains, some strains may mainly contribute to acidification, while other strains mainly contribute to the viscosity, which can be an indication of the texture. However, the strains within the mixture can influence each other's activity. They can thus depend on each other for their activity and expression of the features in the resulting fermented food product. Furthermore, performance indicators, such as acidification and post-acidification, may be linked. In some advantageous examples, this complexity is handled by using a Bayesian model. The method allows to pick the right combination of individual strains, and to determine their relative abundance (cf. quantitative ratios) in the mixture of microbial strains. Typically, it is unknown how individual strains interact with each other. The Bayesian model does not require explicit knowledge with regard to the interactions between the individual strains. Instead, the interactions of the strains in the mixture are taken into account implicitly.

Although the Bayesian model enables an efficient optimization using the performance indicators, also other methods may be used for optimization in some examples.

Optionally, the at least one performance indicator is associated to at least one desired functional property of the obtained fermented product.

Optionally, the at least one performance indicator includes at least one of: a texture indicator, an acidification indicator, a post acidification indicator or a shelf life indicator.

The selection of the texture, acidification and post acidification performance indicators may provide in a more effective optimization using the statistical model. From experiments it is seen that these performance indicators may provide sufficient information for the model to perform the optimization efficiently. Furthermore, they can be measured using an integrated automated system, providing for a high-throughput experimentation.

The texture indicator may be determined by means of measurements relating to viscosity. It may be easy and reliable to use viscosity measurements. Advantageously, viscosity can be determined by an automated high throughput system. Other rheological parameters may also be used for determining an indication of the texture. For example, spinning the fermented food may allow determining a measure of the fluidity, which is also related to viscosity. Other performance indicators may also be used, for example an indicator which can be indicative of a durability of added pro-biotics during the fermentation process. Pro-biotics may be added to a milk fermentation. The survival of pro-biotics during fermentation may be important. In some examples, a performance indicator is employed related to bioprotective activity. A higher bioprotective activity may also increase the shelf life. In some examples, one of the performance indicators is indicative of visual characteristics of the fermented food product (e.g. coloring, browning, glossiness, reflectivity, etc.). Another example of a performance indicator is a time period needed to reach a target acidification. Another example of a performance indicator is a flavor profile or the like.

Optionally, a measure of grittiness is used as the texture indicator. This may be easily determined using imaging systems. In this way, the texture performance indicator can be determined more quickly and efficiently.

Optionally, the at least one performance indicator is associated to a fermentation process parameter, such as for instance acidification speed. Other process parameter can be also used as the performance indicator.

In some examples, process profiles, process responses and/or process conditions related to one or more fermentation process parameter are monitored and employed as performance indicator. Various process parameters/variables can be taken into account. A combination of fermentation process parameters may also be used.

The process profiles may include time series data, for example values indicative of sugar, ethanol, other related compounds, temperature, pH profiles, etc., monitored during the fermentation process. Furthermore, setpoints or target values may be provided for the process. The process profiles may also include process responses, e.g. the amount of ethanol production may be used as a performance indicator of the fermentation process. It will be appreciated that various other responses of the process exist, for example glycerine levels, cell count, amount of yeast used in the process, etc.

It will be appreciated that the fermentation process parameter may refer to process data sets including for a plurality of time points a value indicative for at least one of: a sugar consumption, ethanol production, pH value, reaction temperature, composition of biomass, enzyme composition, yeast cell count, or glycerol production. Various other examples are possible.

Optionally, the measurements for determining data indicative of the at least one performance indicator can be performed online. For example, acidification speed measurements can be obtained by means of online sensors which enable real-time monitoring.

Optionally, the statistical model is further configured to determine quantitative composition ratios of additive mixtures to be used with respective selected microbial strain mixtures, the additive mixture including at least two additives selected from a group of additives.

Various additives may also impact the performance of the microbial strain mixture. Therefore, significantly better results can be obtained by taking the additive mixtures also into account. The microbial strain mixtures and the additive mixtures can be tuned together, since they can have significant influence on each other.

Optionally, the additives in the group of additives are selected based on their effect on the at least one performance indicator.

The sensitivity of the microbial strains to additives may be determined, and based on the sensitivities the additives having the most impact on the performance of the functionalities of the strains can be selected in the group of additives.

Optionally, the group of additives is chosen such that only additives having an influence on the at least one performance indicator are included. In this way, the number of iterations/experiments needed can be significantly reduced. Hence, a more efficient optimization process can be achieved.

Optionally, the group of additives comprises at least one of: enzymes, vitamins, metabolites, or chemicals.

Optionally, the enzymes include at least one of a group consisting of: lactase(s), (microbial) rennets such as chymosin, lipase(s), phospholipase(s), glucose exidase, β-galactosidase, protease(s), transglutaminase and other cross-linking enzymes.

It is seen that some of these enzymes, such as for instance lactases, may have a relatively strong influence on the at least one performance indicator. Lactase hydrolyzes the milk sugar lactose into glucose and galactose and as a result microbial mixture of for instance lactic acid bacteria adapts its metabolism, which affects, in some cases, the acidification rate, the time to reach (TTR) a desired pH, such as pH 4.6 (in case of e.g. yogurt production), the texture or the degree of post-acidification during shelf-life. The extent to which these properties are changed depend on e.g. the dosage of lactase, but also on the species and ratio of strains that are present in the microbial mixture.

Since the additives are taken into account during tweaking/tuning of the quantitative composition ratio of microbial strain mixture, significantly improved end results can be obtained. The additives may influence properties of the fermented food product. This influence can be difficult to predict, as it may depend on the particular microbial strains and their abundances in the microbial strain mixture. Advantageously, the method can effectively take into account such influence during experimentation campaigns.

Optionally, the chemicals include at least one of a group consisting of: hydrocolloids, antifungal compounds such as sorbate and benzoate, nisin, sweeteners, such as Stevia and sucralose, fat replacers, formate, acetate or propionate. Other chemicals may for instance be minerals or trace metals. Such chemicals may also influence the properties of the fermented food product, and taking them into account during tuning of the mixture can reduce the difference between the actual properties and the desired/targeted properties of the fermented food product.

Optionally, the vitamins include at least one of a group consisting of: vitamin A, B1, B2, B3, B5, B6, B7, B9, B12, C, D, E and/or K. In some cases the additives may also have a combined effect which can be effectively taken into account according to the method of the invention.

Optionally, the group of microbial strains, from which microbial strains are selectable for forming microbial strain mixtures with chosen quantitative composition ratios, includes at least 10 different microbial strains, more preferably at least 30 different microbial strains, even more preferably at least 50 different microbial strains.

Advantageously, the group of microbial strains may have a large number of strains from which strains can be selected for composing mixtures. For example, for roughly 300 strains, the total number of possible combinations can be larger than 1090, even not taking into account different possible ratios. It would not be feasible to test such a large number of combinations.

The method provides for an objective way of designing new strain mixtures or cultures for use in the production of fermented food products. Advantageously, the group of strains from which particular strains are selected to form the strain mixtures can be relatively large, providing an improved design freedom in obtaining desired/targeted properties of the fermented food product.

Optionally, multiple performance indicators are determined for each fermentation process, wherein the objective function is based on a combination of the multiple performance indicators.

Defining the objective function may be based on custom preferences and the application. The objective function may be a combination of scaled or normalized performance indicators. The combination may be linear or non-linear.

Optionally, the monitored performance indicators are stored as historical datapoints in a database.

Prior available knowledge based on historical data can be used. The data from historical experiments can be stored for other optimization campaigns. For example, these historical data points can be provided to the Bayesian model. As a result, the time required to reach an optimized microbial strain mixture with targeted properties can be significantly reduced.

Optionally, the first cycle involves using prior knowledge based on historical data fed to the statistical model. Additionally or alternatively, the first cycle may involve picking experiments for exploring the design space.

Optionally, the method is carried out using an autonomous experimentation laboratory.

The autonomous or self-driving lab may allow execution of experiments on an automated robotics platform. The data generated on the platform can be fed to a machine learning framework including the statistical model. The statistical model can determine the experiments to be executed in a next round/cycle in order to iteratively obtain targeted/desired properties of the fermented food products.

Optionally, the method cycles are carried out by means of an automated system, wherein the automated system is provided with an automated robotic arrangement for performing automatic culturing of food product with individual microbial strain mixtures of the selected set of plurality of microbial strain mixtures.

The automated system can provide for a completely autonomous experimentation platform. The performance indicators may be measured using the automated system and provided as input to the statistical model, which can determine new experiments in order to optimize a pre-defined objective function.

Optionally, the automated robotic arrangement comprises one or more handling units configured to, during each method cycle, compose the selected set of plurality of microbial strain mixtures having different composition ratios and distribute said selected set of plurality of microbial strain mixtures over multiple wells of a microplate with the to be fermented food product therein, wherein the fermentation processes during each cycle are carried out in the multiple wells of the microplate.

Optionally, the automated robotic arrangement distributes, during each method cycle, the selected set of plurality of microbial strain mixtures having different composition ratios over a subset of the total number of wells of the microplate, wherein the same microplate is used in a plurality of successive method cycles.

The performance of the strains can be measured using an automated system. In some advantageous examples, performance indicators related to acidification, post-acidification and texture (e.g. by measuring viscosity) can be measured using an automated measurement unit of the automated system. These three performance indicators may provide an efficient optimization. It will be appreciated that the use of other and/or additional performance indicators are also envisaged.

The method may rely on high-throughput measurement techniques. The automated system may be configured to carry out high-throughput evaluation of the selected experiments.

Optionally, the automated system is provided with a mixing unit configured to mix microbial strains for producing microbial strain mixtures with desired quantitative composition ratios.

Optionally, viscosity measurements are carried by a pipetting unit of the automated system. In this way, an indication of the texture can be obtained during pipetting, which results in a highly efficient system.

Optionally, the automated system is provided with a dilution unit configured to dilute microbial strains for producing diluted microbial strain mixtures with desired quantitative composition ratios.

Optionally, the fermented food product is a fermented milk product. The fermented food product may for instance be a dairy product, such as a cheese product or a yogurt product.

Although employing the Bayesian model provides important advantages for determining improved quantitative composition ratios of strain mixtures and optionally additive mixtures, especially due to the enormous amounts of possible combinations and the limited resources for performing actual experiments, it is also possible to use other statistical models. Optionally, the statistical model incorporates at least one of the following: linear regression, logistic regression, kernel ridge regression, decision trees, hidden Markov models, support vector machines, neural networks, reinforcement-based learning, cluster-based learning, hierarchical clustering, genetic algorithms, response surface modelling, surrogate modelling or combinations thereof.

A Bayesian optimization model may significantly reduce the time required to reach a novel microbial strain mixture/blend with targeted/desired properties. In combination with the use of selected performance indicators (cf. texture, acidification and/or post-acidification), a more efficient way of determining improved/enhanced strain mixtures with particular quantitative composition ratios which provide the fermented food product with the desired/target properties can be obtained.

According to an aspect, a method of determining a quantitative composition ratio of a microbial strain mixture for use in a fermentation process, is provided, wherein in a first step, product properties for the fermented food product to be produced are defined. In a second step, m out of n strains are selected to form different strain mixtures with different composition ratios. In a third step, the cultures are assembled with different biomass abundances, using an automated system such as a robotics platform. In a fourth step, the fermentation process using the assembled cultures are carried out and performances are tracked. In a fifth step, a statistical model is employed which is configured to propose the strains and their biomass abundances to be tested in the next round based on the measured performances of the previous iteration(s), going back to the third step. After a number of iterations, the strains and their biomass abundances that lead to desired product properties can be determined.

Advantageously, the product quality can be improved by exploring large design spaces. By employing a Bayesian model as the statistical model, less resources may be required for determining strain mixtures which can provide for desired product properties of the fermented food product. As a result, the time-to-market can be significantly shortened.

The method can be used for identifying microbial strain mixtures with desired properties, or well-suited for fermenting a food product into a fermented food product with desired properties.

According to an aspect, the invention provides for an automated system for performing autonomous experimentation, wherein the automated system comprises a controller configured to carry out the method according to the invention.

The automated system provides an efficient way to determine new strain combinations leading to desired product properties. This enables a fast response to market needs.

Optionally, the automated system is configured to inoculate selected strain mixtures on a well plate or microplate (e.g. 24 well plate, 96 well plate, 384 well plate, etc.). The well plate may have a matrix of wells in which one strain mixture and a food product can be introduced. In each well a different strain mixture (cf. different strains and/or strains with different ratios) can be introduced. It will be appreciated that other experimentation tools may also be used, for instance an experimentation cartridge, experimentation strips, experimentation containers, experimentation chambers, etc. The system may perform high-throughput experimentation, which are performed iteratively.

According to an aspect, the invention provides for a computer program product containing a set of instructions stored thereon that, when executed by a controller of the system according to the invention, results in the system performing a method according to the invention.

The computer program product may be useful for designing and developing microbial strain mixtures/blends for producing fermented food products with desired functional properties. Advantageously, the computer program product model may employ a Bayesian model. In this way, the process to obtain microbial strain mixtures/blends usable in a process for producing a fermented food product with desired/targeted functional properties can be speeded up significantly.

According to an aspect, the invention provides for a method for producing a fermented food product, comprising: determining a quantitative composition ratio of a microbial strain mixture according to the method of the invention, and applying the determined microbial strain mixture with said quantitative ratio in a fermentation process.

According to an aspect, the invention provides for a use of a microbial strain mixture with an optimized quantitative composition ratio determined by employing the method according to the invention, in a process for producing a fermented food product.

The group of strains may include a large number strains such as, but not limited to, Bifidobacterium, Lactobacillus, Streptococcus thermophilus, Lactococcus, Propionibacterium, etc.

It will be appreciated that a strain may be a genetic variant within a biological species. It will be appreciated that a mixture or blend of strains may be a set of strains with a particular quantitative composition ratio (cf. relative abundances of the set of strains).

It will be appreciated that the quantitative composition ratio may relate to the dosages of individual strains within the mixture of strains, hence the relative abundance. Although selecting a subset of strains from the group of strains may be a discrete problem, determining the quantitative composition ratios may be considered as a continuous problem.

It will be appreciated that a wide variety of different performance indicators may be used. The performance indicator may be application dependent.

It will be appreciated that microbial may refer to micro-organisms. Examples are bacteria, yeast, fungi, etc. For example, the microbial strains may be bacterial strains used in a fermentation process of a food product.

It will be appreciated that food product is to be interpreted broadly. It may refer to any biological product. For example, the food product may be milk, which can result in a fermented food product which can be consumed by humans. However, the food product may also refer to biological products such as grass (cf. animal food product).

It will be appreciated that repeatedly may be interpreted to refer to performing at least two iterations, more preferably at least 5 iterations or more.

Viscosity measurements can be performed using a viscometer. The viscometer may be configured to perform viscosity measurement on an undisturbed product. In some examples, the viscometer is configured to determine viscosity by measuring the force required to turn a spindle into the product at a given rate. In some examples, the viscometer employs a T-C spindle for measuring an indication of the viscosity of non-flowing thixotropic material (gels, cream). The viscometer may be arranged to slowly lower or raise a rotating T-bar spindle into the sample so that not always the same region of the sample is sheared (helical path).

Although the procedures of for example the methods and processes described herein may be described in a particular order for ease of description, unless the context dictates otherwise, various procedures may be reordered, added, and/or omitted in accordance with various embodiments. Furthermore, the procedures described with respect to one method or process may be incorporated within other described methods or processes. Likewise, system components described according to a particular structural architecture and/or with respect to one system may be organized in alternative structural architectures and/or incorporated within other described systems. Therefore, while various embodiments are described with or without certain features for ease of description and to illustrate exemplary aspects of those embodiments, the various components and/or features described herein with respect to a particular embodiment can be substituted, added and/or subtracted from among other described embodiments, unless the context dictates otherwise. Consequently, although several exemplary embodiments are described, it will be appreciated that the invention is intended to cover all modifications and equivalents within the scope of the following claims.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any 3, 4, 5, >6 or >7 etc. of said members, and up to all said members.

It will be appreciated that any of the aspects, features and options described in view of the method apply equally to the system and the described computer program product. It will also be clear that any one or more of the above aspects, features and options can be combined.

### BRIEF DESCRIPTION OF THE FIGURES

The invention will further be elucidated on the basis of exemplary embodiments which are represented in a drawing. The exemplary embodiments are given by way of non-limitative illustration. It is noted that the figures are only schematic representations of embodiments of the invention that are given by way of non-limiting example.

### In the Drawing:

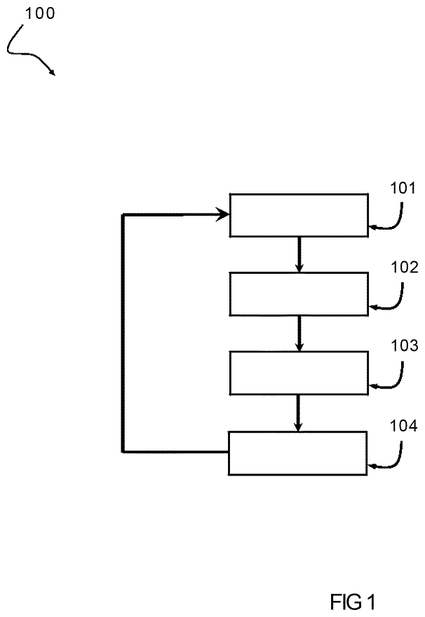

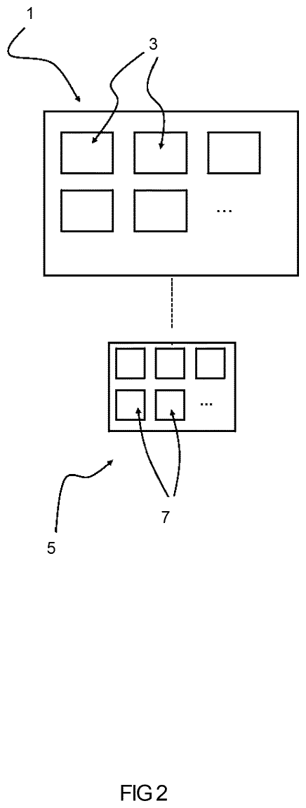

FIG. 1 shows a schematic diagram of a method;

FIG. 2 shows a schematic diagram of an example of mixture selection;

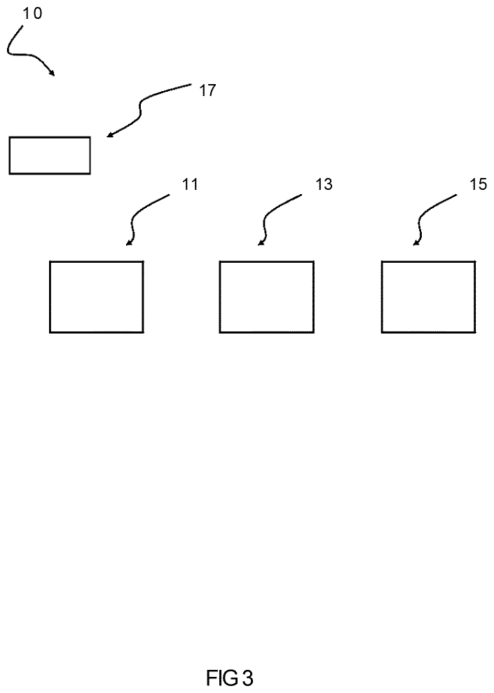

FIG. 3 shows a schematic diagram of an embodiment of a system;

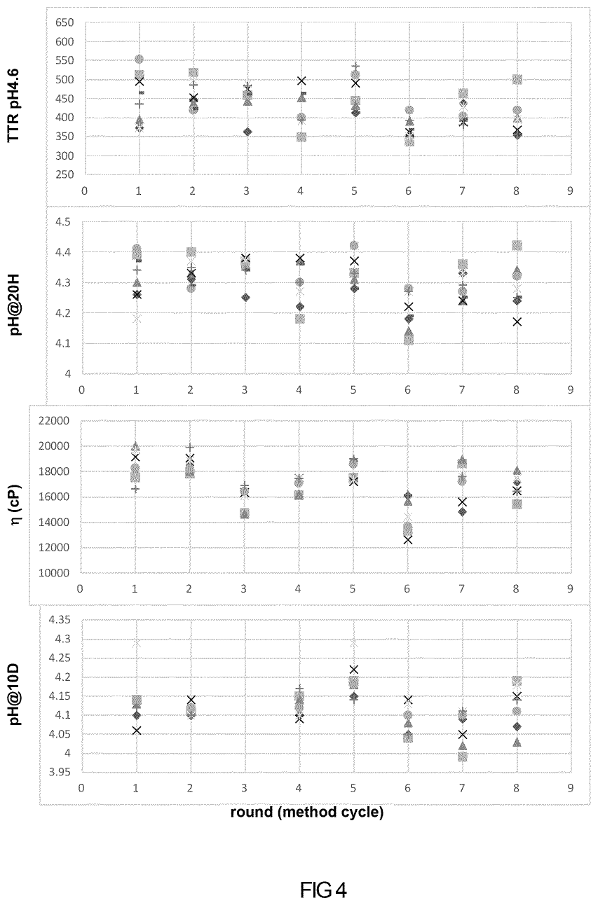

FIG. 4 shows illustrates values of performance indicators for exemplary mixtures; and

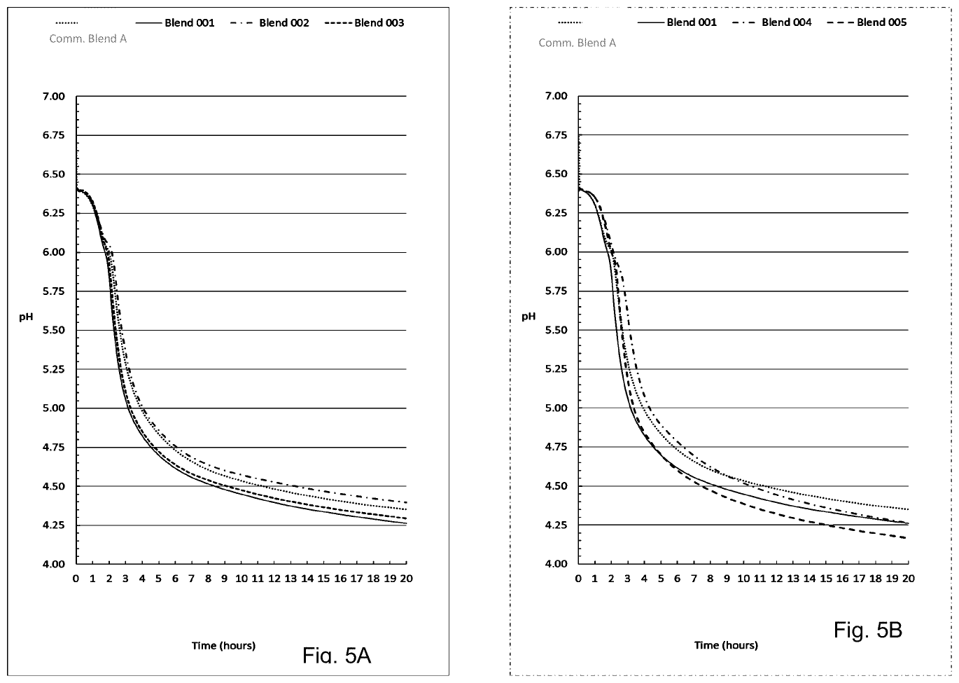

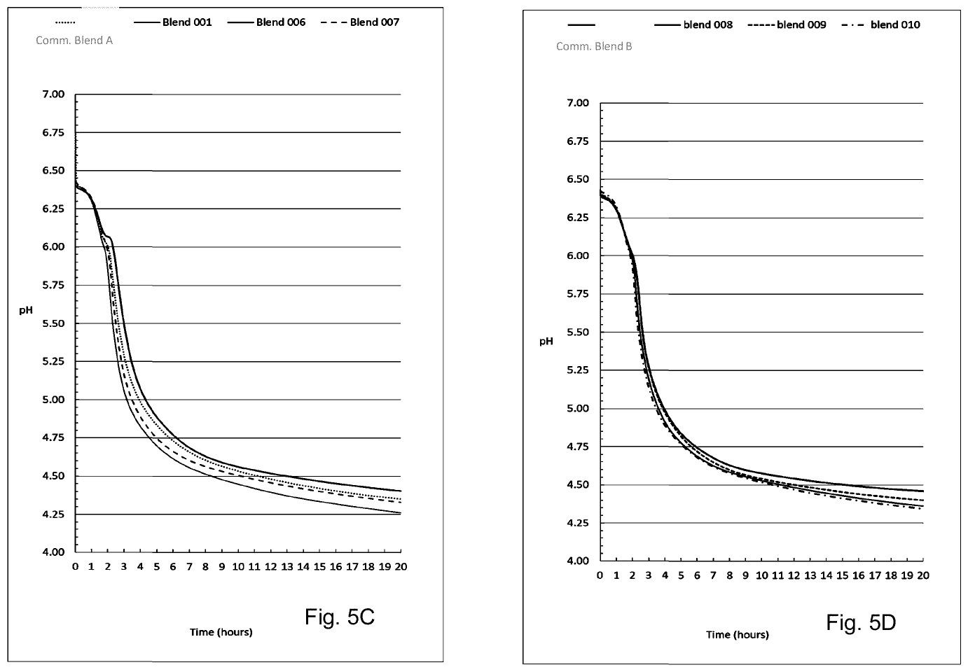

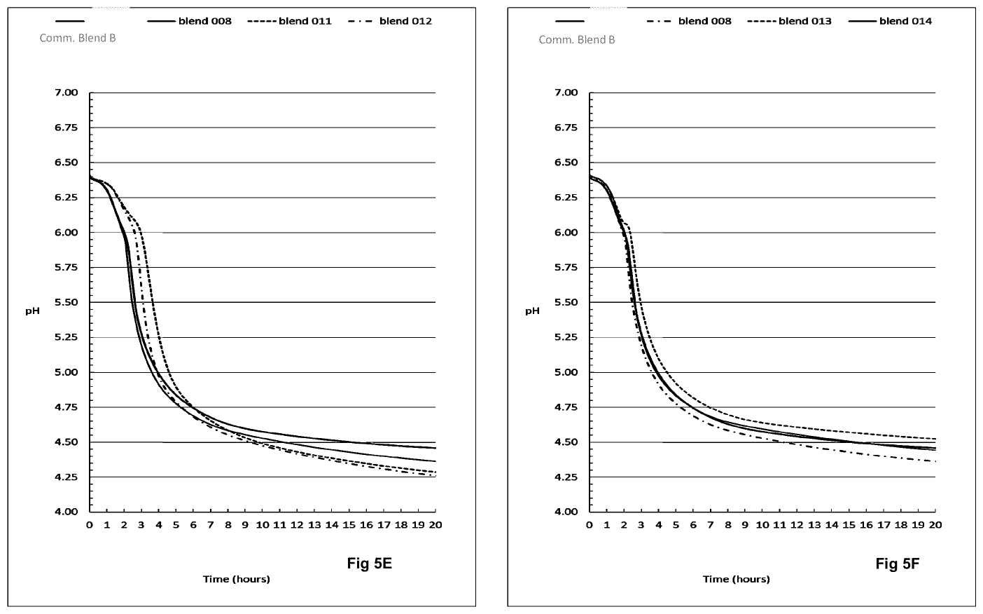

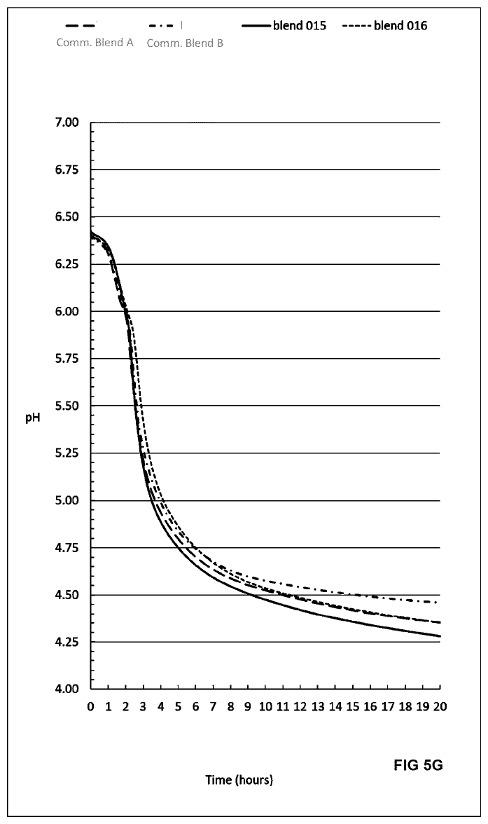

FIGS. 5A to 5G illustrate exemplary Acidification profiles for exemplary mixtures.

### DETAILED DESCRIPTION

FIG. 1 shows a schematic diagram of a method 100 of determining a quantitative composition ratio of a microbial strain mixture for use in a fermentation process. The microbial strain mixture includes at least two microbial strains selected from a group of microbial strains. In a first step 101, a set of plurality of microbial strain mixtures having different quantitative composition ratios is selected. In a second step 102, fermentation processes for each microbial strain mixture of the selected set of plurality of microbial strain mixtures having different quantitative composition ratios are concurrently performed. The fermentation processes may involve culturing a food product with different microbial strain mixtures of the selected set of plurality of microbial strain mixtures for obtaining a fermented food product. In a third step 103, at least one performance indicator of the microbial strain is determined, for each of the fermentation processes. The at least one performance indicator may be associated to at least one desired (functional) property of the obtained fermented product, and/or a fermentation process parameter, such as for instance acidification speed. In a fourth step 104, the at least one performance indicator for each of the fermentation processes is provided to a statistical model which is configured to optimize a predefined objective function, wherein the statistical model is configured to determine, based on said determined performance indicators, a subsequent set of plurality of microbial strain mixtures with different quantitative composition ratios for selection in a next cycle, cf. feedback the selection to the first step 101. The above steps may form a method cycle. The method cycles can be repeatedly carried out until one or more mixtures are identified which can be used for producing fermented food products with targeted/desired (functional) properties. Additionally or alternatively, a maximum number of iterations may be defined in this optional step. It is also possible to stop repeating the process when no further improvement could be obtained (cf. not converging).

### Exemplary Use-Cases are Provided Below

### 1) Optimize Inoculation Dosages of a Given Blend.

Let a blend B consist of a set of strains SB={s1, s2, . . . , sm} with the corresponding inoculation dosages X={x1, x2, . . . , xm}. The goal is to determine X in such a way that the objective function f=f (Y) is maximized where Y denotes the performance indicator vector.

### 2) Optimize Blend Composition and the Respective Inoculation Dosages Given a Set of Strains.

Given a set of strain S={s1, s2, . . . , sn}, the goal is to determine a blend B consisting of a set of strains SB={s1, s2, . . . , sm} where SB is a subset of S along with their along with the inoculation dosages X={x1, x2, . . . xm} in such a way that the objective function f=f (Y) is maximized where Y denotes the KPI vector.

### 3) Optimize Blend Composition and the Respective Inoculation Dosages Given a Set of Strains as Well as the Media Composition.

Given a set of strain S={s1, s2, . . . , sn} and a list of media components M={m1, m2, . . . , mk}, the goal is to determine a blend B consisting of a set of strains SB={s1, s2, . . . , sm} where SB is a subset of S along with their along with the inoculation dosages X={x1, x2, . . . , xm} and media components MB={m1, m2, . . . , ml} in such a way that the objective function f=f (Y) is maximized where Y denotes the KPI vector.

The selection of the same strains may still lead to different product properties, if the same strains have different amounts in the strain mixture. Hence, the strain composition ratio can have significant influence on the properties of the fermented food product. The quality of the fermented food product depends on the culture composition. The method allows to efficiently select the subset of strains out of the group of strains, and their corresponding abundance in the mixture for providing the food product with the predefined desired properties.

In some examples, the statistical model is a Bayesian model. Advantageously, less resources are required by employing the Bayesian model. A relatively low number of cycles may be needed in order to determine enhanced or optimized microbial strain mixtures. As a result, the development costs and time-to-market can be significantly reduced.

FIG. 2 shows a schematic diagram of an example of a mixture selection. Each selected microbial mixture 1 may include a subset of different strains 3 of a group of strains, with a particular relative quantitative composition ratio. In this example, also additive mixtures 5 are associated to each microbial strain mixture 1. The additives 7 in the additive mixture 5 are selected from a group of additives. The additive mixtures 5 are to be used with the respective microbial strain mixtures 1. Advantageously, the group of additives from which the additives are selected may be chosen based on the effect of the additives on the at least one performance indicator. In this way, the statistical model can also be used to optimize the additives for use with the microbial strain mixtures. Hence, not only the composition of the strains is tuned, but also the additives such as vitamins, lactases, enzymes, etc. For instance, certain enzymes such as lactase may have a significant influence on the chosen performance indicators such as texture, acidification and post-acidification. Hence, taking such additive also into account during optimization of the microbial strain composition ratio provides significant improvements to the final product.

The method allows the use of a relatively large group of microbial strains from which the mixtures are composed, for example the group may include more than hundred different strains. The strains in the group of microbial strains can be different strains and/or variants of the same strain or same species. The group of strains from which particular strains are selected and combined to form the strain mixtures can provide a natural diversity which can be explored and exploited for obtaining particular desired targeted properties of the fermented food product.

The method may be employed for identifying strain mixtures/cultures which can provide desired properties of the resulting fermented food, the desired properties for instance relating to at least one of: limited post acidification, reduced browning, desired flavor/taste, desired texture and/or substance properties, prolonged shelf life, etc.

In some examples, a self-driving lab is utilized for performing design-build-test-learn cycles, wherein the build and test phases are carried out on the robotics platform and the thereby generated data, by performing the tests and measuring the performances, are processed and fed to the statistical model to propose a new round of experiments.

In some examples, the statistical model is a Bayesian model, which can effectively perform the tasks faster, whilst reducing the required resources as a limited number of iterations are required. In this way, the process of identifying novel enhanced microbial strain mixtures can be made significantly less expensive.

FIG. 3 shows a schematic diagram of an embodiment of a system 10 for determining a quantitative composition ratio of a microbial strain mixture for use in a fermentation process. In some examples, the system 10 is a self-driving experimentation laboratory configured to provide a completely autonomous experimentation platform.

The system 10 comprises an automated robotic arrangement 11 for performing automatic culturing of food product with individual microbial strain mixtures of the selected set of plurality of microbial strain mixtures. The automated robotic arrangement 11 may include one or more handling units configured to, during each method cycle, compose the selected set of plurality of microbial strain mixtures having different composition ratios and distribute said selected set of plurality of microbial strain mixtures over multiple wells of a microplate with the to be fermented food product therein, wherein the fermentation processes during each cycle are carried out in the multiple wells of the microplate.

The system 10 further includes an automated measurement unit 13 for monitoring at least one performance indicator. In some examples, the measurement unit 13 is configured to track acidification, post-acidification and texture (e.g. by measuring viscosity). These three performance indicators may enable an efficient and robust optimization. Additionally or alternatively, other performance indicators may be used.

The system 10 further includes a mixing unit 15 configured to mix microbial strains for producing microbial strain mixtures with desired quantitative composition ratios.

The system 10 further includes a controller 17 arranged to control the different components of the system, such as the robotic arrangement 11, automated measurement unit 13, and the mixing unit 15, in order to carry out the method cycles. More particularly, the controller may be configured to repeatedly perform method cycles, each method cycle comprising the steps of: selecting a set of plurality of microbial strain mixtures having different quantitative composition ratios; concurrently performing fermentation processes for each microbial strain mixture of the selected set of plurality of microbial strain mixtures having different quantitative composition ratios, wherein the fermentation processes in each cycle involve culturing a food product with each microbial strain mixture of the selected set of plurality of microbial strain mixtures for obtaining a fermented food product; determining, for each of the fermentation processes, at least one performance indicator, associated to at least one desired functional property of the obtained fermented product and/or to one or more fermentation process parameters; and providing the at least one performance indicator for each of the fermentation processes to a statistical model which is configured to optimize a predefined objective function, wherein the statistical model is configured to determine/recommend, based on said determined performance indicators, a subsequent set of plurality of microbial strain mixtures with different quantitative composition ratios for selection in a next cycle.

Examples of performance indicators associated to at least one functional property of the obtained fermented product are: a texture indicator, an acidification indicator, a post acidification indicator or a shelf life indicator. Advantageously, these performance indicators can result in a high throughput, whilst providing efficient optimization. Additionally or alternatively, the at least one performance indicator is associated to a fermentation process parameter, such as for instance acidification speed. It has been observed that acidification speed can also be implemented in a high throughput automated system, whilst providing efficient optimization capabilities. However, other fermentation process parameters, such as fermentation time can also be employed.

In some examples, the at least one performance indicator can be a certain value measured during the fermentation process which is to be optimized (e.g. data indicative of acidification speed, fermentation time, growth rate, maximum production, lag-phase, etc.). Such output values can be regularly monitored during the fermentation process. Employing such process parameters as the performance indicator may enable improvement of the fermentation process. Advantageously, this can result in significantly more efficient processes, saving a lot of operational time. This can in turn lead to improved properties of the fermented food product.

The system may provide for a closed-loop experimentation platform for determining most suitable strain mixture selection which provides desired/targeted product properties. The experiments may be carried out fully automatically on a robotics platform. The generated data can be fed to a machine learning framework which proposes new experimental designs. The strain selection can be improved iteratively, such that it converges to a suitable strain mixture which in a fermentation process can result in a food product with desired properties.

If strains are selected from a group of hundreds of strains corresponding to the available production strains, a very large number of combinations are possible. It is not feasible to test every combination. Furthermore, a larger variety of possible strain mixtures are possible if the different possible ratios are taken into account. By employing a Bayesian model, the optimization of the microbial strain mixture composition ratio can be performed faster and at a lower cost. Advantageously, enhanced strain mixtures can be more efficiently identified, and the time to market of novel fermented food products with desired properties can be significantly reduced.

The method cycles may be carried out iteratively. For each subsequent iteration, a subsequent set of plurality of microbial strain mixtures with different quantitative composition ratios for selection in a next cycle may be determined by means of the statistical model, based on said determined performance indicators. The successive experiments can be performed on wells of a well plate for example. In some examples, a subset of wells on a well plate are used per iteration. However, it is also envisaged that each well of a well plate is used in each iteration, and that for each iteration a new well plate is used. The invention can provide for a model-based design and/or optimization of a microbial strain mixture compositions for use in fermentation processes, for example for producing a fermented product based on an initial biological product. The fermented product may be a fermented food product. A tailored design/optimization can be achieved taking into performance indicators. Different optimization algorithms can be employed for performing the optimization of the quantitative composition ratio of the microbial strain mixture for use in the process involving fermentation. In some advantageous examples, a Bayesian optimization is carried out.

Fermentation in food processing is a process wherein carbohydrates are converted into organic acids or alcohol, or other fermentation (by-) products, with the use of microorganisms under aerobic, semi-aerobic or anaerobic conditions. Fermentation is often performed with microbial strains, such as yeasts or bacteria. Almost any food product can be fermented, such as milk, olives, beans, grains, fruit such as grapes, honey, fish, meat and tea, and/or extracts thereof.

A variety of bacterial genera are used for fermentation, for example Streptococcus, Acetobacter, Bacillus, Bifidobacterium, Lactobacillus etc. Lactobacillus for example, is able to convert sugars into lactic acid, and therefore actively lowering the pH of its environment. Some Lactobacillus species can be used as starter cultures for a high variety of fermented products, such as yogurt, cheese, sauerkraut, pickles, beer, cider, kimchi, cocoa, kefir and other fermented foods.

Acidification can be performed with a wide range of bacteria, such as Lactococcus lactis ssp. lactis and Lactococcus lactis ssp. cremoris, Streptococcus salivarius ssp, thermophiles, Lactobacillus helveticus, Propionibacter shermani, etc.

In some examples, the process is performed for producing a milk-based product such as cheese or yogurt.

Milk can be from an animal source, e.g. cow, goat, sheep, buffalo, etc. Additionally, milk can also have a non-dairy source, such as plant milk. Examples of plant milk include almond milk, coconut milk, rice milk, soy milk, etc.

It will be appreciated that the method may include computer implemented steps. All above mentioned steps can be computer implemented steps. Embodiments may comprise computer apparatus, wherein processes performed in computer apparatus. The invention also extends to computer programs, particularly computer programs on or in a carrier, adapted for putting the invention into practice. The program may be in the form of source or object code or in any other form suitable for use in the implementation of the processes according to the invention. The carrier may be any entity or device capable of carrying the program. For example, the carrier may comprise a storage medium, such as a ROM, for example a semiconductor ROM or hard disk. Further, the carrier may be a transmissible carrier such as an electrical or optical signal which may be conveyed via electrical or optical cable or by radio or other means, e.g. via the internet or cloud.

Some embodiments may be implemented, for example, using a machine or tangible computer-readable medium or article which may store an instruction or a set of instructions that, if executed by a machine, may cause the machine to perform a method and/or operations in accordance with the embodiments.

Various embodiments may be implemented using hardware elements, software elements, or a combination of both. Examples of hardware elements may include processors, microprocessors, circuits, application specific integrated circuits (ASIC), programmable logic devices (PLD), digital signal processors (DSP), field programmable gate array (FPGA), logic gates, registers, semiconductor device, microchips, chip sets, et cetera. Examples of software may include software components, programs, applications, computer programs, application programs, system programs, machine programs, operating system software, mobile apps, middleware, firmware, software modules, routines, subroutines, functions, computer implemented methods, procedures, software interfaces, application program interfaces (API), methods, instruction sets, computing code, computer code, et cetera. Herein, the invention is described with reference to specific examples of embodiments of the invention. It will, however, be evident that various modifications, variations, alternatives and changes may be made therein, without departing from the essence of the invention. For the purpose of clarity and a concise description features are described herein as part of the same or separate embodiments, however, alternative embodiments having combinations of all or some of the features described in these separate embodiments are also envisaged and understood to fall within the framework of the invention as outlined by the claims. The specifications, figures and examples are, accordingly, to be regarded in an illustrative sense rather than in a restrictive sense. The invention is intended to embrace all alternatives, modifications and variations which fall within the spirit and scope of the appended claims. Further, many of the elements that are described are functional entities that may be implemented as discrete or distributed components or in conjunction with other components, in any suitable combination and location.

In the claims, any reference signs placed between parentheses shall not be construed as limiting the claim. The word ‘comprising’ does not exclude the presence of other features or steps than those listed in a claim. Furthermore, the words ‘a’ and ‘an’ shall not be construed as limited to ‘only one’, but instead are used to mean ‘at least one’, and do not exclude a plurality. The mere fact that certain measures are recited in mutually different claims does not indicate that a combination of these measures cannot be used to an advantage.

### EXAMPLES

The invention will now be demonstrated in the following, non-limiting examples. The examples are used to demonstrate the use of the inventive method for determining a quantitative composition ratio of a microbial strain mixture in a fermentation process for the manufacture of yoghurt. As the inventive method can be applied on any type of blend, the specific blends used in this example are less relevant. It will be appreciated that the method as disclosed herein can also be used to advantage for other microbial food product processes, in particular processes having mixtures of strains which have a complex interaction and influence on final product be obtained. Process wherein the method as disclosed herein may be used to particular advantage include, but are not limited to: microbial ethanol production processes, e.g. fermented alcoholic beverages such as wine or beer and/or microbial process for production of vegan plant-based dairy altematives, such as plant-based yoghurt or kefir.

### Strains and Products

TABLE 1
details as to the strains and products as referenced herein.

| Strain or | | |
|---|---|---|
| Culture | Species Composition | Obtainable from |
| Comm. | Streptococcus thermophilus | DSM Food and Beverages, |
| Blend A | Lactobacillus delbrueckii | Delft, the Netherlands |
| | subsp. bulgaricus | |
| Comm. | Streptococcus thermophilus | DSM Food and Beverages, |
| Blend B | Lactobacillus delbrueckii | Delft, the Netherlands |
| | subsp. bulgaricus | |
| Comm. | Streptococcus thermophilus | DSM Food and Beverages, |
| Blend C | Lactobacillus delbrueckii | Delft, the Netherlands |
| | subsp. bulgaricus | |
| Comm. | Streptococcus thermophilus | DSM Food and Beverages, |
| Blend D | Lactobacillus delbrueckii | Delft, the Netherlands |
| | subsp. bulgaricus | |
| CBS141584 | Lactobacillus rhamnosus | CBS141584 was deposited |
| | | at the Centraalbureau |
| | | voor Schimmelcultures, |
| | | Utrecht, the Netherlands |
| CBS116412 | Lactobacillus paracasei | CBS116412 was deposited |
| | | at the Centraalbureau |
| | | voor Schimmelcultures, |
| | | Utrecht, the Netherlands |
| CBS148322 | Lactobacillus casei | CBS148322 was deposited |
| | | at the Centraalbureau |
| | | voor Schimmelcultures, |
| | | Utrecht, the Netherlands |
| CBS148323 | Lactobacillus rhamnosus | CBS148323 was deposited |
| | | at the Centraalbureau |
| | | voor Schimmelcultures, |
| | | Utrecht, the Netherlands |
| ATCC34905 | P. roqueforti | ATCC (www.atcc.org) |
| ATCC18110 | Debaryomyces hansenii | ATCC (www.atcc.org) |

### Milk Fermentation

12% (w/v) RSM (reconstituted skim milk) was pasteurized by heating in a water bath for 15 minutes at 90° C., followed by 30 minutes at 85° C. After pasteurization, the milk was quickly cooled in ice-water and kept at 4° C.

The pasteurized milk was inoculated with the cultures as indicated in the Examples, and incubated at 42° C. The pH was continuously monitored using a CINAC apparatus (Ysebaert, France). Alternatively, acidification was performed in microplates using fluorescent pH probes for measuring the acidification. Several methods have been published and are known to those skilled in the art.

Simultaneously, for the purpose of additional measurements, samples have been prepared in an identical manner. For instance, in cups (150 ml volume, diameter 5.5 cm), or in microplates. Samples for additional analysis (post-acidification, texture analysis, challenge tests for bioprotective activity) were harvested when the pH of the fermented milk reached pH 4.6. The fermented milks thus obtained were cooled and stored at 4° C. until further analysis (yogurt samples).

### Brookfield Viscosity Measurement

Viscosity measurements were performed using a Brookfield RVDVII+Viscometer, which allows viscosity measurement on an undisturbed product (directly in the pot). The Brookfield Viscometer determines viscosity by measuring the force required to turn the spindle into the product at a given rate. The Helipath system with a T-C spindle was used as it is designed for non-flowing thixotropic material (gels, cream). It slowly lowers or raises a rotating T-bar spindle into the sample so that not always the same region of the sample is sheared (helical path). Thus, the viscometer measures constantly the viscosity in fresh material, and is thus thought to be the most suitable for measuring stirred yogurt viscosity. A speed of 30 rpm was used for 31 measuring points, at an interval of 3 sec. The average of the values between 60 and 90 seconds are reported. All viscosity measurements were performed at least in duplicate.

### Post-Acidification Measurement

The degree of post-acidification was determined by measurement of the pH of the yogurt samples. To this end, the yogurt samples were incubated at different temperatures to mimic storage of the product, at 4° C., 7° C. and or 20° C. (see specific Examples). For each time point, at each temperature, a separate small sample (1-10 mL) is being prepared, which was discarded after the pH measurement. The results were plotted in a graph.

### Challenge Test

The fermented milk products were subjected to a challenge test, to show the bioprotective activity of the bioprotective adjuncts CBS141584, and/or CBS116412, and/or CBS148322 and/or CBS148323. Each sample was divided: one part was not contaminated; one part was contaminated with mould spores of ATCC34905. Optionally, in some experiments (see Examples) one part was contaminated with yeast cells ATCC18110. The contaminants were added to the fermented milk products at about 50 mould spores per cup (P. roqueforti ATCC34905) or 50 yeast cells per milliliter (Debaryomyces hansenii ATCC18110). Both species are well-known dairy contaminants in the industry.

A mild homogenization was applied in order to mix the yeast cells through the yogurts after the yeast addition. The fungal spores were pipetted on top of the yogurt samples after filling the cups (about 125 grams per cup) and the tubes (about 20 grams per tube).

Experiments were also performed in microplates (MTP), using lower volumes (200 μL-2 mL) and contaminants were added proportionally to the larger volumes.

The cups or microplates were closed with appropriate lids and stored at the desired temperature (for instance, 20° C.; see Examples). Cups were inspected and photographed at regular intervals (days, weeks; see Examples) to look for the occurrence of mould growth on the surface of the products, up to 8 weeks. For yeast growth monitoring, yeast cell counts were done by serial dilution of the samples and plating on OGY-agar (Sigma Aldrich), which is selective for yeasts.

### Example 1—the Initial Design

The experiment was set out to find the optimal ratio of different lactic acid bacteria (i.e. in this case S. thermophilus and L. bulgaricus for the production of yogurt, and L. rhamnosus for providing bioprotective activity, thereby extending the shelf-life of the yogurt).

The starting point was the selection of four different commercially available starter cultures for yogurt (as illustrated in Table 2), including Commercial Blend A and Commercial Blend B, which are both blends of two strains of S. thermophilus (ST) and one strain of L. bulgaricus (LB). The composition of both blends is partially similar, concerning specific strains used in the blend, but there are also differences (i.e. differences in dosages and strains used in the blend), as illustrated in the table below, wherein ST=Streptococcus thermophilus; LB=Lactobacillus delbrueckii subsp. bulgaricus.

TABLE 2
Composition of strains (the amounts as indicated are intended to be used per 1000 L)

| | Strain 1 | | Strain 2 | | Strain 3 | Strain 4 | | Strain 5 | Strain 6 |
|---|---|---|---|---|---|---|---|---|---|
| | (ST) | | (ST) | | (ST) | (LB) | | (ST) | (LB) |
| | (relative | | (relative | | (relative | (relative | | (relative | (relative |
| Blend * | abundance) | | abundance) | | abundance) | abundance) | | abundance) | abundance) |
| Commercial | 48 | g | 28.8 | g | | 8 | g | | |
| Blend A | | | | | | | | | |
| Commercial | 45.6 | g | | | 19.2 g | 1.6 | g | | |
| Blend B | | | | | | | | | |
| Commercial | | | 9.6 | g | | 8 | g | 48 g | |
| Blend C | | | | | | | | | |
| Commercial | | | 47.04 | g | | | | | 2 g |
| Blend D | | | | | | | | | |

* The Commercial Blends are herein also abbreviated as “Comm. Blend”.

Strains 1 through 6 were obtained by isolation from their respective commercial sources using methods and techniques known in the art. Each strain contributes to the properties of the final product but also influences contributions of other strains in the mixture in a complex and typically non-linear fashion.

Two blends are mild, comm, blend A and comm, blend B, which means that the degree of post-acidification (i.e., acidification during shelf-life) is perceived to be relatively low. Commercial Blend A is perceived to have a higher degree of viscosity compared to Commercial Blend B.

An experimental design was prepared in which variations on the above dosages were applied. In addition, a commercial bioprotective adjunct culture (CBS141584) was added, or not, at the recommended dosage of 38 grams/1000L. Other bioprotective strains were included as well, such as for instance CBS116412, and/or CBS148322 and/or CBS148323.

The aim of this specific example was to illustrate the effect of variations in the blend compositions on the a) acidification rate, b) degree of post-acidification, c) the viscosity, and d) shelf-life extension.

The experimental design was as described in the table below. The composition of each blend is given in grams per 1000 liters (L) of milk.

TABLE 3A
composition of used blends

| | Strain 1 | Strain 2 | Strain 3 | Strain 4 | CBS141584 |
|---|---|---|---|---|---|
| Blend | (g/1000 L) | (g/1000 L) | (g/1000 L) | (g/1000 L) | (g/1000 L) |
| Comm. | 48 | 28.8 | | 8 | |
| Blend A | | | | | |
| Blend 01 | 48 | 28.8 | | 8 | 38 |
| Blend 02 | 48 | 28.8 | | 1.6 | |
| Blend 03 | 48 | 28.8 | | 1.6 | 38 |
| Blend 04 | 4.8 | 28.8 | | 8 | |
| Blend 05 | 4.8 | 28.8 | | 8 | 38 |
| Blend 06 | 48 | 4.8 | | 8 | |
| Blend 07 | 48 | 4.8 | | 8 | 38 |
| Comm. | 45.6 | | 19.2 | 1.6 | |
| Blend B | | | | | |
| Blend 08 | 45.6 | | 19.2 | 1.6 | 38 |
| Blend 09 | 45.6 | | 19.2 | 8 | |
| Blend 10 | 45.6 | | 19.2 | 8 | 38 |
| Blend 11 | 4.8 | | 19.2 | 1.6 | |
| Blend 12 | 4.8 | | 19.2 | 1.6 | 38 |
| Blend 13 | 45.6 | | 6 | 1.6 | |
| Blend 14 | 45.6 | | 6 | 1.6 | 38 |
| Blend 15 | 24 | 14.4 | 9.6 | 4.8 | |
| Blend 16 | 24 | 14.4 | 9.6 | 4.8 | 38 |

Yogurts were prepared at 100 ml scale by inoculating 12% RSM (reconstituted skimmed milk) with the commercial starter cultures and blends derived thereof, described in the table above. Each acidification experiment was performed in duplicate. In addition, multiple plastic cups were filled with the same composition.

The 100 ml samples were incubated at 42° C. in a water bath. The evolution of the pH in time was followed using a CINAC device (http://www.amsalliance.com/product/icinac) during at least 20 hours. The plastic cups were simultaneously incubated in the same water bath.

Once the pH of a certain blend reached pH 4.6, the corresponding plastic cups were removed from the water bath. The cups were cooled in ice water for 15 minutes (0° C.) and subsequently stored overnight at 4° C. for further analysis.

In FIGS. 5A to 5G depicts the acidification of the blends, whereby the averages of two duplicate measurements are depicted in the graph. FIG. 5A shows Acidification profiles (pH) of Commercial Blend A, Blend 01, Blend 02 and Blend 03 as a function of time, at 42° C. in 12% RSM. FIG. 5B shows Acidification profiles (pH) of blends Commercial Blend A, Blend 01, Blend 04 and Blend 05 as a function of time, at 42° C. in 12% RSM. FIG. 5C shows Acidification profiles (pH) of Commercial Blend A, Blend 01, Blend 06 and Blend 07 as a function of time, at 42° C. in 12% RSM. FIG. 5D shows Acidification profiles (pH) of blends Commercial Blend B, Blend 08, Blend 09 and Blend 10 as a function of time, at 42° C. in 12% RSM. FIG. 5E shows Acidification profiles (pH) of blends Commercial Blend B, Blend 08, Blend 11 and Blend 12 as a function of time, at 42° C. in 12% RSM. FIG. 5F shows Acidification profiles (pH) of blends Commercial Blend B, Blend 08, Blend 13 and Blend 14 as a function of time, at 42° C. in 12% RSM. FIG. 5G Acidification profiles (pH) of Commercial Blend A, Commercial Blend B, Blend 15 and Blend 16 as a function of time, at 42° C. in 12% RSM.

As shown in FIG. 5A, the four acidification curves display a similar progress for about two hours; from that point on, the four curves start to exhibit different acidification profiles. Adding bioprotective culture CBS141584 to Commercial Blend A (Blend 01) results in a faster acidification. Blend 02, which has a 5x lower amount of Strain 4 than Commercial Blend A, exhibits a somewhat slower acidification rate relative to Commercial Blend A. Blend 03, with 38 grams/1000L CBS141584 added and a lower amount of Strain 4, is faster than blend Commercial Blend A, but is somewhat slower than Blend 01. In summary, it can be concluded that lowering the amount of Strain 4 resulted in a slower acidification rate, while adding CBS141584 to the blend results in a faster acidification rate.

From each of the curves, feature points were extracted, which describe the progress of the acidification in a numerical way. An important feature point is TTR pH 4.6 (Time To Reach (in minutes) a pH value of 4.6), the approximate pH at which the yogurt fermentation is terminated in industrial practice. Also, pH @ 20h (the pH after 20 hours of fermentation) is an important feature point, as it correlates with the post-acidification (PA) during shelf-life. These values are listed in Table 3B.

The target value of the TTR pH 4.6 is a value close to the value of the reference culture, Commercial Blend A, with a two-sided range of 30 minutes. Likewise, the target value for the pH @ 20h was set at the target value of the reference culture Commercial Blend A, in which a lower pH value of 0.05 pH units was accepted. A higher pH value was considered to be preferred.

Minor changes are usually observed between experiments, due to natural experimental variation.

Therefore, comparisons to the reference (Commercial Blend A) are made within one experimental design.

The samples in the plastic cups were used to determine the viscosity (cP) using a Brookfield RVDVII+Viscometer. The average of three measurements is reported in Table 3B as well. The target range for this key performance indicator is greater than 17000 cP or target cP >[reference CP-500].

The pH during shelf-life at 20° C. was measured (post-acidification, or PA). The measured values at day 10 are presented in Table 3B as well. The target range is to obtain a pH value as close as possible to the reference culture, or better. The lowest acceptable delta-pH compared to the reference culture Commercial Blend A is 0.05 pH units. In other words: target PA>[reference PA-0.05].

Also here, natural experimental variation is known to exist between experiments.

Shelf-life extension was measured by performing a challenge test as described above. The target value is an extended shelf life compared to the reference culture Commercial Blend A. If the shelf-life was extended compared to the reference culture, the shelf-life extension was scored with a “+”. If the shelf-life extension was not reached, i.e. spoilage occurred at the same day or earlier as compared to reference culture Commercial Blend A, the score “−” was given. In Example 1, shelf-life extension was analyzed using ATCC34905 only.

TABLE 3B
initial experimental design and resulting data on four KPIs (TTR pH 4.6 (in minutes),
pH @ 20 h, viscosity (in cP), PA (pH at day 10 at 20° C.) and shelf-
life extension (relative to the reference culture Commercial Blend A). The composition
of the blends in the table are provided in grams per 1000 L of milk.

| | | | | | | TTR | pH | | PA | |
|---|---|---|---|---|---|---|---|---|---|---|
| | Strain | Strain | Strain | Strain | | pH 4.6 | @ | Viscosity | Day | Shelf-life |
| Culture | 1 | 2 | 3 | 4 | CBS141584 | (min) | 20 h | (cP) | 10 | extension |
| Comm. | 48 | 28.8 | | 8 | | 460 | 4.34 | 18853 | 4.25 | − |
| Blend A | | | | | | | | | | |
| Blend001 | 48 | 28.8 | | 8 | 38 | 372 | 4.26 | 17808 | 4.1 | + |
| Blend002 | 48 | 28.8 | | 1.6 | | 512 | 4.39 | 18740 | 4.26 | − |
| Blend003 | 48 | 28.8 | | 1.6 | 38 | 394 | 4.3 | 17533 | 4.14 | + |
| Blend004 | 4.8 | 28.8 | | 8 | | 494 | 4.26 | 20007 | 4.13 | − |
| Blend005 | 4.8 | 28.8 | | 8 | 38 | 372 | 4.18 | 19161 | 4.06 | + |
| Blend006 | 48 | 4.8 | | 8 | | 552 | 4.41 | 19661 | 4.29 | − |
| Blend007 | 48 | 4.8 | | 8 | 38 | 435 | 4.34 | 18278 | 4.14 | + |
| Comm. | 45.6 | | 19.2 | 1.6 | | 552 | 4.45 | 17199 | 4.38 | − |
| Blend B | | | | | | | | | | |
| Blend008 | 45.6 | | 19.2 | 1.6 | 38 | 464 | 4.37 | 16619 | 4.12 | + |
| Blend009 | 45.6 | | 19.2 | 8 | | 496 | 4.41 | 18236 | 4.3 | − |
| Blend010 | 45.6 | | 19.2 | 8 | 38 | 446 | 4.35 | 17665 | 4.12 | + |
| Blend011 | 4.8 | | 19.2 | 1.6 | | 460 | 4.29 | 14582 | 4.17 | − |
| Blend012 | 4.8 | | 19.2 | 1.6 | 38 | 430 | 4.26 | 13999 | 4.07 | + |
| Blend013 | 45.6 | | 6 | 1.6 | | 742 | 4.52 | 20296 | 4.39 | − |
| Blend014 | 45.6 | | 6 | 1.6 | 38 | 576 | 4.44 | 19138 | 4.14 | + |
| Blend015 | 24 | 14.4 | 9.6 | 4.8 | | 402 | 4.28 | 20243 | 4.2 | − |
| Blend016 | 24 | 14.4 | 9.6 | 4.8 | 38 | 497 | 4.34 | 18176 | 4.1 | + |

On basis of the results obtained, novel blends were proposed that need to meet the above mentioned criteria (performance indicators) with respect to acidification rate (TTR pH 4.6), pH @20h, viscosity, shelf-life extension and PA (pH at day 10, at 20° C.). Also, constraints with respect to dosages of the single strains in the blends can be part of the novel blend considerations. Those novel blends were proposed by applying machine learning frameworks as described in Example 2 (Bayesian optimization). Subsequently, the proposed novel blends were tested in a new cycle of experiments.

### Example 2—Bayesian Optimization

All designs listed herein were generated using Bayesian optimization, except for the designs in Experiment 1 (see Table 3A). The Experiment 1 served as starting point of the blend optimization. For subsequent cycles also strains from Commercial Blend C and Commercial Blend D were introduced into the design space. First, all KPIs are normalized with respect to their mean value for a given reference (here: Commercial Blend A) across all available experiments to account for experiment-to-experiment variation which is always faced when dealing with biological data. Then inventors used sklearn.pipeline.Pipeline (sklearn version 1.0.2) to create a Pipeline object for each KPI. Thereby, each Pipeline is initialized with a (name, transformer) tuple; the name is the name of the KPI and the transformer a sklearn.gaussian_process.GaussianProcessRegressor object. As Kernels inventors selected the sum of sklearn.gaussian_process.kernels.RBF and sklearn.gaussian_process.kernels. WhiteKernel; for the RBF Kernel inventors thereby choose the arguments length_scale to be the number of features worked with and length_scale_bounds=(1e-05, 1e2), respectively. Furthermore, inventors set n_restarts_optimizer=9 and normalize_y=True for the GaussianProcessRegressor.

Then, the dataset will test be split set using into a training and sklearn.model_selection.train_test_split with random_state=0; for the remaining arguments inventors used the default settings. For each of the Pipeline objects inventors now called the .fit method using the training set as input. Invnetors then applied the .predict method to all of the Pipeline objects using the test set as input.

Now that models for all of our KPIs are available inventors started writing the optimization algorithm. For this inventors provided a way to calculate fitness for a query point by calculating the left or right p value for each KPI. Hereafter, a hill climber was started to find the optimal combination of ingredients/process conditions to reach our KPIs. To do so, inventors defined two constraints: First, inventors specified the blend to always contain least 1 ST and 1 LB. Second, inventors specified the blend to contain at least one bioprotective strain. The hill climber starts from all existing blends and allows for the following permutations: an ingredient can be added with a random amount which is chosen from a uniform distribution between 0.2 and 0.5, and ingredient can be removed, the amount of an ingredient can be increased by a random amount which is chosen from a uniform distribution between 0 and 0.5, the amount of an ingredient can be decreased by a random amount which is chosen from a uniform distribution between 0 and 0.5. An individual hill climber is then implemented for each design as previously tested in previous rounds. If in none of the hill climbers an improved fitness can be observed, the optimization stops. The recipes that were determined are then ordered according to their fitness and the best n recipes are chosen for the next round of experiments; n thereby depends on the experimental throughput that is possible per iteration.

### Example 3-Further Blend Testing

Using the approach described in Example 2, several rounds of novel blend testing were performed. The cycle consists of three steps: generation of data (step 1; see e.g. Example 1); using the generated experimental data of step 1 to feed into the statistical model (step 2); prediction of novel combinations of strains by the statistical model (step 3). Step 3 will be followed by testing the novel designs (step 1, again).

Table 3 depicts the composition and key performance indicators of the initial design, Tables 4 to 11 depict the composition and key performance indicators of the design for subsequent method cycles 2 to 8.

TABLE 4
designs of cycle 2 (all strains as indicated are in grams/1000 L milk)

| | | | | | | TTR | pH | | | shelf |
|---|---|---|---|---|---|---|---|---|---|---|
| | Strain | Strain | Strain | Strain | | pH | @ | | Day | life |
| Culture | 1 | 2 | 3 | 4 | CBS141584 | 4.6 | 20 h | cP | 10 | extension |
| Blend001 | 48 | 28.8 | | 0.16 | 38 | 446 | 4.31 | 18497 | 4.1 | + |
| Blend008 | 45.6 | 0 | 19.2 | 0.032 | 38 | 518 | 4.4 | 17073 | 4.15 | + |
| Blend017 | 48 | 25.92 | 17.4 | 0.032 | 38 | 437 | 4.33 | 17845 | 4.11 | + |
| Blend018 | 45.12 | 4.8 | 19.2 | 0.576 | 7.6 | 452 | 4.33 | 18012 | 4.11 | + |
| Blend019 | 45.6 | 23.04 | 19.2 | 0.032 | 7.6 | 497 | 4.37 | 19055 | 4.14 | + |
| Blend020 | 45.6 | 11.52 | 19.2 | 0.768 | 15.2 | 418 | 4.28 | 18863 | 4.1 | + |
| Blend021 | 48 | 28.8 | | 0.04 | 19 | 486 | 4.35 | 18193 | 4.12 | + |
| Blend022 | 48 | 48 | | 0.264 | 7.6 | 422 | 4.29 | 19920 | 4.1 | + |
| Blend023 | 40.32 | 23.52 | | 0.032 | 38 | 438 | 4.32 | 18327 | 4.09 | + |
| Blend024 | 48 | 10.08 | | 0.048 | 36.48 | 583 | 4.4 | 19188 | 4.11 | + |
| Blend025 | 45.6 | | 19.2 | 0.8 | 29.26 | 400 | 4.27 | 18248 | 4.07 | + |
| Blend026 | 48 | | 24 | 0.176 | 7.6 | 474 | 4.37 | 17043 | 4.14 | + |
| Blend027 | 45.6 | | 19.2 | 0.128 | 10.64 | 514 | 4.39 | 19003 | 4.13 | + |
| Blend028 | 45.6 | | 19.2 | 0.8 | 38 | 388 | 4.26 | 16712 | 4.06 | + |
| Comm. | 45.6 | | 19.2 | 0.032 | | 554 | 4.44 | 18288 | 4.38 | − |
| Blend B | | | | | | | | | | |
| Comm. | 48 | 28.8 | | 0.16 | | 554 | 4.39 | 20040 | 4.23 | − |
| Blend A | | | | | | | | | | |

TABLE 5
designs of cycle 3 (all strains as indicated are in grams/1000 L milk)

| | | | | | | TTR | pH | | |
|---|---|---|---|---|---|---|---|---|---|
| | Strain | Strain | Strain | Strain | | pH | @ | | shelf life |
| Culture | 1 | 2 | 3 | 4 | CBS141584 | 4.6 | 20 h | cP | extension |
| Blend001 | 48 | 28.8 | | 8 | 38 | 362 | 4.25 | 16405 | + |
| Blend008 | 45.6 | | 19.2 | 1.6 | 38 | 458 | 4.37 | 14485 | + |
| Blend029 | 24 | | 24 | 1.6 | 3.8 | 442 | 4.36 | 14718 | + |
| Blend030 | 28.8 | | 24 | 1.6 | 7.6 | 474 | 4.38 | 14648 | + |
| Blend031 | 33.6 | | 18 | 4 | 7.6 | 488 | 4.37 | 16358 | + |
| Blend032 | 38.4 | | 18 | 8 | 5.7 | 458 | 4.35 | 16105 | + |
| Blend033 | 20.16 | | 16.2 | 5.6 | 7.6 | 482 | 4.34 | 16438 | + |
| Blend034 | 33.6 | 4.8 | | 8 | 7.6 | 461 | 4.34 | 16892 | + |
| Blend035 | 48 | 9.6 | | 12 | 7.6 | 410 | 4.3 | 17160 | + |
| Blend036 | 9.12 | 1.92 | | 5.2 | 7.6 | 480 | 4.3 | 17760 | + |
| Blend037 | 19.68 | 5.28 | 16.2 | 4 | 7.6 | 464 | 4.31 | 16750 | + |
| Blend038 | 29.28 | 12 | 10.8 | 4.4 | 7.6 | 466 | 4.34 | 16815 | + |
| Blend039 | 28.8 | | | 8 | 7.6 | 494 | 4.35 | 16790 | + |
| Blend040 | 28.8 | | | 4 | 7.6 | 546 | 4.4 | 17178 | + |
| Comm. | 45.6 | | 19.2 | 1.6 | | 488 | 4.42 | 15305 | − |
| Blend B | | | | | | | | | |
| Comm. | 48 | 28.8 | | 8 | | 395 | 4.3 | 16788 | − |
| Blend A | | | | | | | | | |

TABLE 6
designs of cycle 4 (all strains as indicated are in grams/1000 L milk)

| | | | | | | TTR | pH | | | shelf |
|---|---|---|---|---|---|---|---|---|---|---|
| | Strain | Strain | Strain | Strain | | pH | @ | | Day | life |
| Culture | 1 | 2 | 3 | 4 | CBS141584 | 4.6 | 20 h | cP | 10 | extension |
| Blend001 | 48 | 28.8 | | 8 | 38 | 350 | 4.22 | 17198 | 4.1 | + |
| Blend005 | 4.8 | 28.8 | | 8 | 38 | 348 | 4.18 | 17855 | 4.07 | + |
| Blend008 | 45.6 | | 19.2 | 1.6 | 38 | 452 | 4.37 | 16120 | 4.15 | + |
| Blend014 | 45.6 | | 6 | 1.6 | 38 | 496 | 4.38 | 16200 | 4.14 | + |
| Blend016 | 24 | 14.4 | 9.6 | 4.8 | 38 | 391 | 4.27 | 17472 | 4.09 | + |
| Blend017 | 48 | 25.92 | 17.4 | 1.6 | 38 | 399 | 4.3 | 17438 | 4.1 | + |
| Blend023 | 40.32 | 23.52 | | 1.6 | 38 | 393 | 4.3 | 17082 | 4.12 | + |
| Blend026 | 48 | | 24 | 8.8 | 7.6 | 462 | 4.36 | 17440 | 4.17 | + |
| Blend036 | 9.12 | 1.92 | | 5.2 | 7.6 | 477 | 4.3 | 18243 | 4.11 | + |
| Blend038 | 29.28 | 12 | 10.8 | 4.4 | 7.6 | 482 | 4.36 | 18488 | 4.15 | + |
| Blend040 | 28.8 | | | 4 | 7.6 | 634 | 4.44 | 17647 | 4.16 | + |
| Comm. | 45.6 | | 19.2 | 1.6 | | 508 | 4.43 | 17123 | 4.37 | − |
| Blend B | | | | | | | | | | |
| Comm. | 48 | 28.8 | | 8 | | 408 | 4.31 | 18335 | 4.22 | − |
| Blend A | | | | | | | | | | |

TABLE 7
designs of cycle 5 (all strains as indicated are in grams/1000 L milk)

| | | | | | | | | TTR | pH | | | shelf |
|---|---|---|---|---|---|---|---|---|---|---|---|---|
| | Strain | Strain | Strain | Strain | | | | pH | @ | | Day | life |
| Culture | 1 | 2 | 3 | 4 | CBS148323 | CBS116412 | CBS148322 | 4.6 | 20 h | cP | 10 | extension |
| Blend041 | 48 | 28.8 | | 8 | 30 | | | 412 | 4.28 | 18805 | 4.15 | + |
| Blend042 | 48 | 28.8 | | 8 | | 6 | | 444 | 4.33 | 18763 | 4.15 | + |
| Blend043 | 48 | 28.8 | | 8 | | | 55 | 431 | 4.31 | 17505 | 4.19 | + |
| Blend045 | 45.6 | | 19.2 | 1.6 | 30 | | | 490 | 4.37 | 17527 | 4.18 | + |
| Blend046 | 45.6 | | 19.2 | 1.6 | | 6 | | 512 | 4.42 | 17212 | 4.22 | + |
| Blend047 | 45.6 | | 19.2 | 1.6 | | | 55 | 512 | 4.42 | 17365 | 4.29 | + |
| Blend048 | 24 | 7.92 | | 8 | 5.1 | | | 534 | 4.33 | 18557 | 4.18 | + |
| Blend049 | 40.08 | 40.08 | | 13.4 | | 2.4 | | 412 | 4.28 | 18987 | 4.14 | + |
| Blend050 | 7.92 | 24 | | 24 | | | 45.65 | 427 | 4.23 | 19458 | 4.11 | + |
| Blend051 | 24 | | 9.9 | 8 | 24.9 | | | 490 | 4.31 | 18483 | 4.14 | + |
| Blend052 | 40.08 | | 50.1 | 13.4 | | 4.8 | | 392 | 4.29 | 15270 | 4.16 | + |
| Blend053 | 7.92 | | 30 | 24 | | | 22 | 400 | 4.28 | 17217 | 4.12 | + |
| Blend054 | 24 | 24 | 9.9 | 13.4 | 15 | | | 416 | 4.25 | 17688 | 4.12 | + |
| Blend055 | 7.92 | 40.08 | 50.1 | 24 | | 1.02 | | 392 | 4.24 | 16675 | 4.09 | + |
| Blend056 | 40.08 | 7.92 | 30 | 34.6 | | | 45.65 | 380 | 4.25 | 16805 | 4.14 | + |
| Comm. | 48 | 28.8 | | 8 | | | | 438 | 4.33 | 16997 | 4.25 | − |
| Blend A | | | | | | | | | | | | |

TABLE 8
designs of cycle 6 (all strains as indicated are in grams/1000 L milk)

| | | | | | | | | | TTR | pH | | | shelf |
|---|---|---|---|---|---|---|---|---|---|---|---|---|---|
| | Strain | Strain | Strain | Strain | | | | | pH | @ | | Day | life |
| Culture | 1 | 2 | 3 | 4 | CBS141584 | CBS148323 | CBS116412 | CBS148322 | 4.6 | 20 h | cP | 10 | extension |
| Blend001 | 48 | 28.8 | | 8 | 38 | | | | 354 | 4.18 | 16102 | 4.05 | + |
| Blend061 | | 33.6 | 30 | 8 | | 30 | | | 336 | 4.11 | 14100 | 4.02 | + |
| Blend065 | 45.6 | | | 16 | | | 6 | | 390 | 4.14 | 13318 | 4.04 | + |
| Blend070 | 48 | 28.8 | | 8 | | 15 | | 27.5 | 360 | 4.22 | 15663 | 4.08 | + |
| Blend074 | 45.6 | | 60 | 4 | | | 4.2 | 16.5 | 351 | 4.27 | 12617 | 4.14 | + |
| Blend080 | 45.6 | | 19.2 | 1.6 | | 9 | 1.8 | 16.5 | 418 | 4.28 | 14443 | 4.13 | + |
| Blend094 | 48 | | 24 | 4 | | | 6 | | 392 | 4.27 | 13670 | 4.1 | + |
| Blend100 | 48 | 38.4 | 24 | 4 | 3.8 | 24 | | | 368 | 4.19 | 16162 | 4.05 | + |
| Blend101 | | 38.4 | 24 | 4 | 3.8 | 15 | 2.4 | | 374 | 4.11 | 16077 | 4.03 | + |
| Blend108 | | 26.4 | | 22 | | 16.5 | 3.3 | | 343 | 3.99 | 17008 | 3.95 | + |
| Blend112 | 48 | 41.76 | | 8 | | 4.8 | 0.9 | 50.6 | 338 | 4.19 | 15813 | 4.09 | + |
| Blend117 | 6.24 | | 58.2 | 6 | | 22.2 | 1.32 | | 356 | 4.13 | 9642 | 4.06 | + |
| Blend126 | 37.92 | | 49.8 | 8 | | 30 | 0.78 | | 330 | 4.16 | 12573 | 4.09 | + |
| Comm. | 45.6 | | 19.2 | 1.6 | | | | | 420 | 4.33 | 14732 | 4.36 | − |
| Blend B | | | | | | | | | | | | | |
| Comm. | 48 | 28.8 | | 8 | | | | | 380 | 4.19 | 16378 | 4.2 | − |
| Blend A | | | | | | | | | | | | | |

TABLE 9
designs of cycle 7(all strains as indicated are in grams/1000 L milk)

| | Strain | Strain | Strain | Strain | Strain | Strain | | |
|---|---|---|---|---|---|---|---|---|
| Culture | 1 | 2 | 3 | 4 | 5 | 6 | CBS141584 | CBS148323 |
| Blend128 | 44.16 | | 21 | 1.6 | | | | 4.8 |
| Blend141 | 45.6 | | 19.2 | 1.6 | | | | 6 |
| Blend285 | | 9.6 | | 8 | 48 | | 38 | |
| Blend287 | | 9.6 | | 8 | 48 | | | 4.8 |
| Blend300 | 45.6 | 1.44 | 21 | 1.6 | | | | 1.5 |
| Blend302 | 48 | 19.2 | | 8 | | 15.5 | | 3 |
| Blend303 | 48 | 28.8 | 9.6 | | | 4.5 | | 9.6 |
| Blend305 | 48 | 28.8 | | 8 | | 17 | | 0.3 |
| Comm. | | 9.6 | | 8 | 48 | | | |
| Blend C | | | | | | | | |
| Comm. | 48 | 28.8 | | 8 | | | | |
| Blend A | | | | | | | | |

| | | | TTR | pH | | | shelf |
|---|---|---|---|---|---|---|---|
| | | | pH | @ | | Day | life |
| Culture | CBS116412 | CBS148322 | 4.6 | 20 h | cP | 10 | extension |
| Blend128 | 0.84 | 49.5 | 436 | 4.33 | 14820 | 4.09 | + |
| Blend141 | 1.2 | 11 | 463 | 4.36 | 15797 | 4.11 | + |
| Blend285 | | | 400 | 4.24 | 18615 | 3.99 | + |
| Blend287 | 0.84 | 49.5 | 388 | 4.24 | 18935 | 4.02 | + |
| Blend300 | 0.3 | 2.75 | 429 | 4.33 | 15613 | 4.05 | + |
| Blend302 | 0.6 | 5.5 | 403 | 4.27 | 17617 | 4.11 | + |
| Blend303 | 0.18 | | 380 | 4.29 | 17210 | 4.1 | + |
| Blend305 | 1.38 | | 395 | 4.25 | 17608 | 4.11 | + |
| Comm. | | | 386 | 4.21 | 18098 | 4.03 | − |
| Blend C | | | | | | | |
| Comm. | | | 383 | 4.3 | 17527 | 4.22 | − |
| Blend A | | | | | | | |

TABLE 10
designs of cycle 8 (all strains as indicated are in grams/1000 L milk)

| | Strain | Strain | Strain | Strain | Strain | Strain | | |
|---|---|---|---|---|---|---|---|---|
| Culture | 1 | 2 | 3 | 4 | 5 | 6 | CBS141584 | CBS148323 |
| Blend001 | 48 | 28.8 | | 8 | | | 38 | |
| Blend128 | 44.2 | | 21 | 1.6 | | | | 4.8 |
| Blend141 | 45.6 | | 19.2 | 1.6 | | | | 6 |
| Blend285 | | 9.6 | | 8 | 48 | | 38 | |
| Blend302 | 48 | 19.2 | | 8 | | 15.5 | | 3 |
| Blend303 | 48 | 28.8 | 9.6 | | | 4.5 | | 9.6 |
| Blend305 | 48 | 28.8 | | 8 | | 17 | | 0.3 |
| Blend340 | 48 | 28.8 | | 8 | | 17 | | 9.6 |
| Blend341 | 48 | 28.8 | 9.6 | | | 4.5 | | 9.6 |
| Blend343 | 48 | 28.8 | | 8 | | | | 30 |
| Blend345 | | 9.6 | | 8 | 48 | | | 30 |
| Comm. | | 9.6 | | 8 | 48 | | | |
| Blend C | | | | | | | | |
| Comm. | 48 | 28.8 | | 8 | | | | |
| Blend A | | | | | | | | |

| | | | TTR | pH | | | shelf |
|---|---|---|---|---|---|---|---|
| | | | pH | @ | | Day | life |
| Culture | CBS116412 | CBS148322 | 4.6 | 20 h | cP | 10 | extension |
| Blend001 | | | 353 | 4.24 | 17178 | 4.07 | + |
| Blend128 | 0.84 | 49.5 | 499 | 4.42 | 15778 | 4.21 | + |
| Blend141 | 1.2 | 11 | 398 | 4.34 | 15407 | 4.19 | + |
| Blend285 | | | 367 | 4.17 | 18087 | 4.03 | + |
| Blend302 | 0.6 | 5.5 | 395 | 4.28 | 16480 | 4.15 | + |
| Blend303 | 0.18 | | 418 | 4.32 | 17458 | 4.18 | + |
| Blend305 | 1.38 | | 355 | 4.25 | 15493 | 4.11 | + |
| Blend340 | 1.02 | | 355 | 4.25 | 16422 | 4.14 | + |
| Blend341 | | | 402 | 4.33 | 17187 | 4.21 | + |
| Blend343 | | | 370 | 4.24 | 16950 | 4.14 | + |
| Blend345 | | | 371 | 4.19 | 18090 | 4.1 | + |
| Comm. | | | 377 | 4.12 | 18682 | 4.1 | − |
| Blend C | | | | | | | |
| Comm. | | | 348 | 4.22 | 16890 | 4.15 | − |
| Blend A | | | | | | | |

That the iterative process yields an optimized blend composition is summarized in Table 11 comparing performance indicators of Blend 001 and Blend340 obtained eight rounds of experimentation.

FIG. 4 illustrates the values of four different performance indicators for the first eight blends of each method cycle (round), where the same marker used in the same cycle indicates the same blend between the different graphs. As shown in Tables 3-10 and the figure illustrate that the iterative approach allows to converge to a combination of desired/targeted properties of the fermented food product. As illustrated individual one of the targeted performance indicators need not converge linearly towards their desired value. Instead the parameter space is explored to discover the optimal combination/blend.

Table 11 summarizes discovery of an optimized blend by the iterative process. The table also shows cycle-to-cycle noise or variations in determined performance indicators (compare e.g. TTR-values of reference Commercial Blend A, Commercial Blend C, and Commercial Blend B across in tables 4-10.

As illustrated in Table 11 Blend001 (provided at the start of the iteration) shows desired speed and shelf life, however, the PA is out of range (delta PA to reference Commercial Blend A in the same cycle >0.05) and also viscosity is too low (delta cP to reference Commercial Blend A in the same cycle >500). Blend340, obtained after eight process cycles, meets all requirements. As shown production speed went up as compared to Blend001. Also delta TTR pH 4.6 to reference Commercial Blend A in the same cycle went from a comparative large negative value to a value smaller than 30 min. The pH @ 20h improved from a comparatively large negative value to a smaller value of 0.03 pH units higher than the reference Commercial Blend A. The delta cP to reference Commercial Blend A in the same cycle is smaller than 500; and the delta PA to reference Commercial Blend A in the same cycle improved from a difference of 0.15 to only 0.01, i.e. within the target of smaller than 0.05.

TABLE 11
selection of summarized results

| | | TTR | Δ | pH | Δ | | Δ | | Δ | |
|---|---|---|---|---|---|---|---|---|---|---|
| | Reference/ | pH | (TTR − | @ | (pH − | Viscosity | (cP − | | (PA − | Shelf-life |
| Cycle | blend | 4.6(min) | TTRref) | 20 h | pH_{ref}) | (cP) | cP_{ref}) | PA | PA_{ref}) | extension |
| 1 | Comm. | 460 | n/a | 4.34 | n/a | 18853 | n/a | 4.25 | n/a | − |
| | Blend A | | | | | | | | | |
| 1 | Blend001 | 372 | −88 | 4.26 | −0.08 | 17808 | −1045 | 4.10 | −0.15 | + |
| 8 | Comm. | 348 | n/a | 4.22 | n/a | 16890 | n/a | 4.15 | n/a | − |
| | Blend A | | | | | | | | | |
| 8 | Blend340 | 355 | 13 | 4.25 | 0.03 | 16422 | −468 | 4.14 | −0.01 | + |

1. A method of determining a quantitative composition ratio of a microbial strain mixture for use in a fermentation process, the microbial strain mixture including at least two microbial strains selected from a group of microbial strains, wherein the method includes repeatedly performing method cycles, each method cycle comprising the steps of:
selecting a set of plurality of microbial strain mixtures having different quantitative composition ratios;
concurrently performing fermentation processes for each microbial strain mixture of the selected set of plurality of microbial strain mixtures having different quantitative composition ratios, wherein the fermentation processes in each cycle involve culturing a food product with each microbial strain mixture of the selected set of plurality of microbial strain mixtures for obtaining a fermented food product;
determining, for each of the fermentation processes, at least one performance indicator of the microbial strain mixture; and
providing the at least one performance indicator for each of the fermentation processes to a statistical model which is configured to optimize a predefined objective function, wherein the statistical model is configured to determine, based on said determined performance indicators, a subsequent set of plurality of microbial strain mixtures with different quantitative composition ratios for selection in a next cycle.

2. The method according to claim 1, wherein the method cycles are repeated one or more times until at least one optimized microbial strain mixture is identified which when used in fermenting of a food product results in a fermented food product having at least one desired functional property.

3. The method according to claim 1, wherein the statistical mode is configured to determine the subsequent set of plurality of microbial strain mixtures with different quantitative composition ratios for selection in a next cycle, based on comparing said determined performance indicators with a respective reference value or range.

4. The method according to claim 1, wherein in one or more, optionally each method cycle,
the selected set of plurality of microbial strain mixtures includes a reference microbial strain mixture having a predetermined composition ratio of microbial strain mixtures;
the fermentation process is concurrently performed for each microbial strain mixture of the selected set of plurality of microbial strain mixtures including the reference microbial strain mixture;
at least one reference performance indicator of the reference microbial strain mixture is determined; and
the at least one reference performance indicator is provided to the statistical model to optimize the predefined objective function, wherein the statistical model is configured to determine the subsequent set of plurality of microbial strain mixtures with different quantitative composition ratios for selection in a next cycle based at least in part on comparing the performance indicators of the different mixtures, respectively, with a value of the at least one reference performance indicator of the reference microbial strain mixture as determined in the same method cycle.

5. The method according to claim 1, wherein the statistical model is a Bayesian optimization model wherein the objective function to be optimized is approximated by a Gaussian process.

6. The method according to claim 1, wherein the at least one performance indicator is associated to at least one desired functional property of the obtained fermented product and includes at least one of: a texture indicator, an acidification indicator, a post acidification indicator or a shelf life indicator.

7. The method according to claim 1, wherein the at least one performance indicator is associated to a fermentation process parameter.

8. The method according to claim 1, wherein the statistical model is further configured to determine quantitative composition ratios of additive mixtures to be used with respective selected microbial strain mixtures, the additive mixture including at least two additives selected from a group of additives.

9. The method according to claim 8, wherein the additives in the group of additives are selected based on their effect on the at least one performance indicator.

10. The method according to claim 8, wherein the group of additives comprises at least one of enzymes, metabolites, vitamins or chemicals.

11. The method according to claim 1, wherein the group of microbial strains, from which microbial strains are selectable for forming microbial strain mixtures with chosen quantitative composition ratios, includes at least 10 different microbial strains, more optionally at least 30 different microbial strains, optionally at least 50 different microbial strains.

12. The method according to claim 1, wherein multiple performance indicators are determined for each fermentation process, wherein the objective function is based on a combination of the multiple performance indicators.

13. The method according to claim 1, wherein the monitored performance indicators are stored as historical datapoints in a database.

14. An automated system for performing autonomous experimentation, wherein the automated system comprises a controller configured to carry out the method according to claim 1.

15. A computer program product containing a set of instructions stored thereon that, when executed by a controller of the system according to claim 14.

16. A method for producing a fermented food product, comprising: determining a quantitative composition ratio of a microbial strain mixture according to claim 1, and applying the determined microbial strain mixture with said quantitative ratio in a fermentation process.

17. A product comprising a microbial strain mixture with an optimized quantitative composition ratio determined by employing the method according to claim 1, in a process for producing a fermented food product.
